(19) United States
(12) Patent Application Publication
(10) Pub. No.: US 2023/0290463 A1
(43) Pub. Date: Sep. 14, 2023

(54) BIOLOGICAL INFORMATION STORAGE SYSTEM AND BIOLOGICAL INFORMATION STORAGE PROGRAM

(71) Applicant: TERUMO KABUSHIKI KAISHA, Tokyo (JP)

(72) Inventor: Satoshi SAWADA, Hadano-shi (JP)

(73) Assignee: TERUMO KABUSHIKI KAISHA, Tokyo (JP)

(21) Appl. No.: 18/322,019

(22) Filed: May 23, 2023

Related U.S. Application Data
(63) Continuation of application No. PCT/JP2021/042796, filed on Nov. 22, 2021.

(30) Foreign Application Priority Data
Nov. 24, 2020 (JP) 2020-194206

(51) Int. Cl. G16H 15/00; A61B 90/00; A61B 5/00; G16H 10/60; G16H 20/40; G16H 50/30

(52) U.S. Cl. CPC G16H 15/00; A61B 90/37; A61B 5/746; G16H 10/60; G16H 20/40; G16H 50/30

(57) ABSTRACT

A biological information storage system includes: a measurement unit that measures biological information of a patient undergoing surgery; a storage unit that stores the biological information measured by the measurement unit; a display unit that displays the biological information; a display processing unit that executes processing of causing the display unit to display a plurality of surgery processes, arranging a plurality of selection processes selected from among the plurality of surgery processes as the scheduled surgery processes in time series in a procedure of the surgery, and causing the display unit to display the plurality of selection processes; a storage processing unit that executes processing of causing the storage unit to store the biological information for each of the selection processes when the surgery is started; and a report generation processing unit that executes processing of generating a report based on the biological information stored in the storage unit.

### CROSS-REFERENCES TO RELATED APPLICATIONS

This application is a continuation of International Application No. PCT/JP2021/042796 filed on Nov. 22, 2021, which claims priority to Japanese Application No. 2020-194206 filed on Nov. 24, 2020, the entire content of both of which is incorporated herein by reference.

### TECHNOLOGICAL FIELD

The present disclosure generally relates to a biological information storage system and a biological information storage program.

### BACKGROUND DISCUSSION

U.S. Patent Application Publication No. 2019/0034591 A discloses a system and method for predicting and summarizing medical events from electronic health records. As in the system disclosed in U.S. Patent Application Publication No. 2019/0034591 A, for example, a system for supporting a medical worker is desired, for example, a system for predicting medical events from electronic health records, automatically recording the electronic health records, or predicting event risks from the electronic health records.

For example, in surgery using various devices such as a heart-lung machine, an anesthesiologist first anesthetizes a patient. Subsequently, medical workers such as a doctor and a nurse prepares for surgery, and a clinical engineer prepares operation for extracorporeal circulation. Then, the anesthesiologist monitors vital signs until the doctor starts the surgery and the extracorporeal circulation is started. When a cannula is inserted and an extracorporeal circulation is ready, the extracorporeal circulation can be started by the clinical engineer in response to the doctor's instruction. When the extracorporeal circulation using the heart-lung machine is performed, the clinical engineer is often responsible for monitoring vital signs and a patient's condition during the extracorporeal circulation. Subsequently, when the doctor stops the operation of the heart-lung machine, the anesthesiologist performs adjustment for removing the heart-lung machine from the patient. For example, in the process of such surgery, the medical workers such as a doctor, a clinical engineer, an anesthesiologist, and a nurse record vital indexes such as a heart rate, a pulse rate, a respiratory rate, a blood pressure, a body temperature, hematocrit (Ht), and hemoglobin (Hb) of the patient, and blood gas indexes such as arterial blood pH, oxygen delivery (DO2), and oxygen consumption (VO2) while communicating with each other.

In this manner, the medical workers need to record the biological information of the patient without omission while performing surgery and communicating with each other. Therefore, a burden on a medical worker during the surgery is relatively great. Furthermore, for example, the biological information of the patient recorded by the anesthesiologist and the biological information of the patient recorded by the clinical engineer may overlap with each other. Furthermore, it is not easy for the doctor to perform medical acts by using a large number of pieces of biological information of a patient in a composite manner. Moreover, for example, blood transfusion is commonly performed in a case where the values of hematocrit and hemoglobin decrease, but the reference values of hematocrit and hemoglobin when determining the execution of the blood transfusion may differ depending on a facility, a doctor in charge, and the like. Therefore, it is desirable to reduce a burden on medical workers such as a doctor, a clinical engineer, an anesthesiologist, and a nurse and to help prevent them from forgetting to record the biological information of the patient.

### SUMMARY

A biological information storage system is disclosed, which is capable of reducing a burden on medical workers such as a doctor, a clinical engineer, an anesthesiologist, and a nurse and preventing the medical workers from forgetting to record biological information of a patient, and a biological information storage program.

According to an aspect of the present disclosure, there is provided a biological information storage system including: a measurement unit that measures biological information of a patient undergoing surgery; a storage unit that stores the biological information measured by the measurement unit; a display unit that displays the biological information; a display processing unit that executes processing of causing the display unit to display a plurality of surgery processes, arranging a plurality of selection processes selected from among the plurality of surgery processes as the scheduled surgery processes in time series in a procedure of the surgery, and causing the display unit to display the plurality of selection processes; a storage processing unit that executes processing of causing the storage unit to store the biological information measured by the measurement unit for each of the selection processes when the surgery is started; and a report generation processing unit that executes processing of generating a report based on the biological information stored in the storage unit.

In the biological information storage system according to the aspect of the present disclosure, the display processing unit causes the display unit to display the plurality of surgical processes. When the medical workers such as a doctor, a clinical engineer, an anesthesiologist, and a nurse select the plurality of the selection processes from among the plurality of surgery processes displayed on the display unit as the scheduled surgery processes, the display processing unit causes the display unit to display the plurality of selected selection processes in time series in the procedure of the surgery. When the surgery is started, the storage processing unit causes the storage unit to store the biological information measured by the measurement unit for each selection process. Since the technique and procedure in surgery are uniform to some extent, the storage processing unit can automatically cause the storage unit to store the biological information of a patient during the surgery along the flow of the surgery. Furthermore, in a case where there is an additional technique each time, the display processing unit executes processing of adding a freely input process to the procedures arranged in time series. The storage processing unit executes processing of causing the storage unit to store the biological information measured by the measurement unit at that time. In this case, a timing at which the storage processing unit causes the storage unit to store the biological information may be an automatic timing, or may be a timing at which the medical worker designates the selection process. The report generation processing unit generates a report based on the biological information stored in the storage unit. Therefore, the report generation processing unit can avoid complicated recording work and automatically create a report with a simple operation. Therefore, the biological information storage system according to the aspect of the present embodiment can reduce a burden on the medical workers such as a doctor, a clinical engineer, an anesthesiologist, and a nurse and prevent them from forgetting to record the biological information of a patient.

In the biological information storage system according to the aspect of the present disclosure, it is preferable that the display processing unit executes processing of adding the selection process selected or input later to the plurality of selection processes selected once, rearranging the plurality of selection processes selected once and the selection process selected or input later in time series in the procedure of the surgery, and causing the display unit to display the rearranged selection processes.

In the biological information storage system according to the aspect of the present disclosure, even after the plurality of selection processes are selected once by the medical worker, in a case where the medical worker adds a selection process later, the display processing unit rearranges the plurality of selection processes selected once and the selection process selected or input later in time series in the procedure of the surgery and causes the display unit to display the rearranged selection processes. Therefore, the biological information storage system according to the aspect of the present disclosure can flexibly cope with various surgery processes.

In the biological information storage system according to the aspect of the present disclosure, it is preferable that after the surgery is started, the display processing unit executes processing of causing the display unit to display the biological information in a designation process designated from among the selection processes that have ended in a progress of the surgery.

In the biological information storage system according to the aspect of the present disclosure, after the surgery is started, the display processing unit causes the display unit to display the biological information in a designation process designated from among the selection processes that have ended in a progress of the surgery. Therefore, the medical worker can easily confirm the biological information in the designation process designated from the ended selection process on the display unit in a shorter time.

In the biological information storage system according to the aspect of the present disclosure, it is preferable that the plurality of selection processes include a predetermined course of the surgery.

In the biological information storage system according to the aspect of the present disclosure, the plurality of selection processes include a predetermined course of the surgery Therefore, the medical worker can save time and effort for individually selecting the scheduled surgery processes and easily select substantially the entire scheduled surgery processes. Furthermore, the surgery process is customized according to differences depending on facilities and doctors, and can be registered.

The biological information storage system according to the aspect of the present disclosure preferably further includes an input processing unit that executes processing of inputting information regarding the patient before the surgery is started.

In the biological information storage system according to the aspect of the present disclosure, the input processing unit inputs information regarding a patient (patient background, preoperative examination data, and the like) before the surgery is started. Therefore, the medical worker can easily confirm, on the display unit, information obtained by combining information regarding a patient before the surgery is started and the biological information stored in the storage unit for each selection process.

The biological information storage system according to the aspect of the present disclosure preferably further includes a risk prediction unit that predicts a risk of a complication that occurs after the surgery is ended based on the biological information stored in the storage unit and a trained model stored in advance in the storage unit.

In the biological information storage system according to the aspect of the present disclosure, the risk prediction unit predicts a complication risk that occurs after the surgery is ended based on the biological information stored in the storage unit for each selection process and the trained model stored in advance in the storage unit. Therefore, the biological information storage system according to the aspect of the present disclosure can present the medical worker with the complication risk that occurs after the surgery is ended, reduce the burden on the medical worker, and support the medical worker's determination.

In the biological information storage system according to the aspect of the present disclosure, it is preferable that the complication includes at least one of acute kidney injury, acute respiratory distress syndrome, cerebral infarction, non-occlusive mesenteric ischemia, postoperative hypotension, or infection.

The biological information storage system according to the aspect of the present disclosure can present the medical worker with a risk of a complication that occurs after the surgery is ended, that is, a risk of a complication including at least one of acute kidney injury, acute respiratory distress syndrome, cerebral infarction, non-occlusive mesenteric ischemia, postoperative hypotension, or infection, reduce a burden on the medical worker, and support the medical worker's determination.

In the biological information storage system according to the aspect of the present disclosure, it is preferable that after the surgery is started, the risk prediction unit future predicts, based on the biological information stored in the storage unit, a future event risk that occurs in a case where a scheduled process designated from among the selection processes which are not still started in a progress of the surgery is performed.

In the biological information storage system according to the aspect of the present disclosure, after the surgery is started, the risk prediction unit further predicts, based on the biological information stored in the storage unit, a future event risk that occurs in a case where the scheduled process is performed. The scheduled process is a process designated from among the selection processes which are not still started in the progress of the surgery. Therefore, the biological information storage system according to the aspect of the present disclosure can present a future event risk that occurs in a case where the scheduled process scheduled to be performed along the flow of the selection process is performed, reduce a burden on the medical worker, and support the medical worker's determination.

In the biological information storage system according to the aspect of the present disclosure, it is preferable that after the surgery is started, the risk prediction unit further predicts, based on the biological information stored in the storage unit, a future event risk that occurs in a case where an unscheduled process different from the selection process is performed.

In the biological information storage system according to the aspect of the present disclosure, after the surgery is started, the risk prediction unit further predicts, based on the biological information stored in the storage unit, a future event risk that occurs in a case where the unscheduled process is performed. The unscheduled process is a process different from the selection process selected as the scheduled surgery process. That is, the unscheduled process is an additionally necessary medical action/process. Therefore, the biological information storage system according to the aspect of the present disclosure can present a future event risk that occurs in a case where the unscheduled process, which is not scheduled to be performed along the flow of the selection process, is performed, reduce a burden on the medical worker, and support the medical worker's determination.

In the biological information storage system according to the aspect of the present disclosure preferably further includes a notification processing unit that executes a process of providing notification of an alert in a case where a probability of the risk predicted by the risk prediction unit is equal to or greater than a threshold value.

In the biological information storage system according to the aspect of the present disclosure, in a case where the probability of the risk predicted by the risk prediction unit is equal to or greater than a threshold value, the notification processing unit provides notification of an alert. Therefore, the medical worker can easily recognize that a risk of a complication that occurs after the surgery is ended, a future event risk that occurs in a case where the scheduled process is performed, and a future event risk that occurs in a case where the unscheduled process is performed are high.

In order to solve the above-described problems, according to another aspect of the present disclosure, there is provided a non-transitory computer-readable medium (CRM) storing a biological information computer program executed by a computer of a biological information storage system including a measurement unit that measures biological information of a patient undergoing surgery, a storage unit that stores the biological information measured by the measurement unit, and a display unit that displays the biological information, the biological information computer program causing the computer to execute a process comprising: causing the display unit to display a plurality of surgery processes; arranging a plurality of selection processes selected from among the plurality of surgery processes as the scheduled surgery processes in time series in a procedure of the surgery and causing the display unit to display the plurality of selection processes; causing the storage unit to store the biological information measured by the measurement unit for each of the selection processes when the surgery is started; and generating a report based on the biological information stored in the storage unit.

According to the biological information computer program according to the aspect of the present disclosure, the plurality of surgical processes are displayed on the display unit. When the medical workers such as a doctor, a clinical engineer, an anesthesiologist, and a nurse select the plurality of the selection processes from among the plurality of surgery processes displayed on the display unit as the scheduled surgery processes, the plurality of selected selection processes are arranged in time series in the procedure of the surgery, and displayed on the display unit. Furthermore, when surgery is started, the biological information measured by the measurement unit is stored in the storage unit for each selection process. Since the technique and procedure in surgery are uniform to some extent, the biological information of a patient during the surgery is automatically stored in the storage unit along the flow of the surgery. Furthermore, in a case where there is an additional technique each time, a freely input process is added to the procedures arranged in time series. Then, the biological information measured by the measurement unit at that time is stored. In this case, a timing at which the biological information is stored in the storage unit may be an automatic timing, or may be a timing at which the medical worker designates the selection process. Then, a report is generated based on the biological information stored in the storage unit. Therefore, it is possible to avoid complicated recording work and automatically create a report with a simple operation. Therefore, the biological information computer program according to the aspect of the present disclosure can reduce a burden on the medical worker such as a surgeon, an anesthesiologist, and a clinical engineer.

According to the present disclosure, there can be provided a biological information storage system capable of reducing a burden on medical workers such as a doctor, a clinical engineer, an anesthesiologist, and a nurse and preventing them from forgetting to record biological information of a patient, and a biological information storage program.

### BRIEF DESCRIPTION OF THE DRAWINGS

FIG. 1 is a block diagram illustrating a configuration of a main part of a biological information storage system according to an embodiment of the present disclosure.

FIG. 2 is a schematic diagram illustrating an example of an image displayed on a touch panel of the present embodiment.

FIG. 3 is a schematic diagram illustrating an example of an image displayed on a touch panel of the present embodiment.

FIG. 4 is a schematic diagram illustrating an example of an image displayed on a touch panel of the present embodiment.

FIG. 5 is a flowchart illustrating a specific example of operation of a biological information storage system according to the present embodiment.

FIG. 6 is a flowchart illustrating a first specific example of processing executed by a risk prediction unit of the present embodiment.

FIG. 7 is a flowchart illustrating a second specific example of processing executed by a risk prediction unit of the present embodiment.

FIG. 8 is a schematic diagram illustrating a third specific example of processing executed by a risk prediction unit of the present embodiment.

### DETAILED DESCRIPTION

Set forth below with reference to the accompanying drawings is a detailed description of embodiments of a biological information storage system and a biological information storage program.

In the drawings, similar components are denoted by the same reference signs, and the detailed description of the similar components will be appropriately omitted.

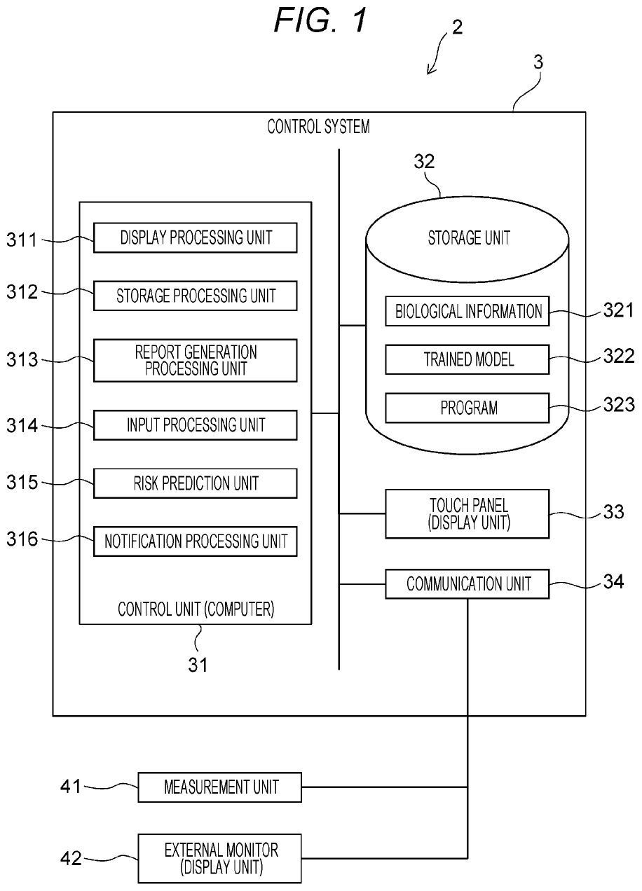

FIG. 1 is a block diagram illustrating a configuration of a main part of a biological information storage system according to an embodiment of the present disclosure.

A biological information storage system 2 according to the present embodiment can include a control system 3 and a measurement unit 41. As illustrated in FIG. 1, the biological information storage system 2 may include an external monitor 42. The external monitor 42 is provided separately from the control system 3, and is an example of a “display unit” of the present disclosure.

The control system 3 acquires various types of information, performs calculation, generates a control signal for controlling the operation of a device such as the external monitor 42, and transmits the control signal to each device. In other words, the control system 3 manages the biological information storage system 2. Details of the control system 3 will be described later. Furthermore, the control system 3 may include a touch panel 33 as an input unit capable of inputting various types of information and as a display unit that displays the various types of information. That is, the “display unit” of the present disclosure may be the external monitor 42 provided separately from the control system 3, or may be the touch panel 33 included in the control system 3. The touch panel 33 is configured to be capable of detecting contact of a finger of an operator such as a medical worker. In the following description, a case where the “display unit” of the present disclosure is the touch panel 33 will be described as an example.

The measurement unit 41 measures biological information of a patient undergoing surgery. Examples of the “biological information” measured by the measurement unit 41 can include a heart rate, a pulse rate, a respiratory rate, a blood pressure, and a body temperature. Furthermore, examples of the “biological information” measured by the measurement unit 41 can include blood test indexes such as lactic acid, hematocrit (Ht), and hemoglobin (Hb), and blood gas indexes such as mixed venous oxygen saturation (SvO2), mixed venous oxygen content (CvO2), carbon dioxide production (VCO2), oxygen delivery (DO2), and oxygen consumption (VO2). Furthermore, in a case where an extracorporeal circulator is used for surgery, examples of the “biological information” measured by the measurement unit 41 can include a circulation perfusion rate, and a cardiac output. Note that the “biological information” measured by the measurement unit 41 is not limited to the examples disclosed herein. The biological information can also be called a parameter.

The “extracorporeal circulation” performed by the extracorporeal circulator includes “extracorporeal circulation” and “assisted circulation”. The extracorporeal circulator can perform both the “extracorporeal circulation” and “assisted circulation”.

The “extracorporeal circulation” means that, for example, in a case where blood circulation in a heart is temporarily stopped in order to perform cardiac surgery, circulation of blood and gas exchange (oxygen addition and/or carbon dioxide removal) for blood are performed by the extracorporeal circulator.

Furthermore, the “assisted circulation” means that the circulation of blood and the gas exchange for blood are also performed by the extracorporeal circulator in a case where the patient's heart for which the extracorporeal circulator is used cannot sufficiently function or in a state in which the gas exchange of the lungs cannot be sufficiently performed.

For example, the extracorporeal circulator is used in a case where the patient's heart does not function normally, or in a case where the patient's heart functions normally but the lungs do not function normally. For example, the extracorporeal circulator can be used in a case where the cardiac surgery for a patient is performed or used for a treatment in an intensive care unit (ICU) after the cardiac surgery. The extracorporeal circulator can perform oxygenator extracorporeal circulation in which a pump of the extracorporeal circulator is operated to remove blood from a vein of the patient, an oxygenator performs gas exchange in the blood to oxygenate the blood, and then the oxygenated blood is returned to an artery or the vein of the patient. The extracorporeal circulator is an apparatus that acts as the heart and lungs.

As described above, the measurement unit 41 measures various biological information of a patient undergoing surgery. Therefore, the measurement unit 41 is not limited to one measurement device, and may include a plurality of measurement devices. The measurement unit 41 measures biological information of a patient undergoing surgery and transmits a signal related to the biological information to the control system 3.

The control system 3 can include a control unit 31, a storage unit 32, a touch panel 33, and a communication unit 34. The control unit 31 can be included in a computer, and reads a program 323 stored in the storage unit 32 to execute various calculations and processing. The “computer” as used herein is not limited to a personal computer, and includes an arithmetic processing device and a microcomputer which are included in an information processing device, and collectively refers to an apparatus and a device which are capable of realizing the functions of the present disclosure with a program. The program 323 of the present embodiment is an example of a “biological information storage program” of the present disclosure.

The control unit 31 can include a display processing unit 311, a storage processing unit 312, a report generation processing unit 313, an input processing unit 314, a risk prediction unit 315, and a notification processing unit 316. The display processing unit 311, the storage processing unit 312, the report generation processing unit 313, the input processing unit 314, the risk prediction unit 315, and the notification processing unit 316 are implemented by the control unit 31 executing the program 323 stored in the storage unit 32. The display processing unit 311, the storage processing unit 312, the report generation processing unit 313, the input processing unit 314, the risk prediction unit 315, and the notification processing unit 316 may be implemented by hardware or may be implemented by a combination of hardware and software.

The display processing unit 311 executes processing of causing the touch panel 33 to display at least one of a surgery process or biological information 321 measured by the measurement unit 41 and stored in the storage unit 32. The display processing unit 311 may execute processing of causing the external monitor 42 to display at least one of the surgery process or the biological information 321. Here, in the present specification, the “process” refers to at least one of a medical act which is performed by the medical worker, for example, such as the doctor, the clinical engineer, the anesthesiologist, and the nurse, a technique, and an event during surgery.

The storage processing unit 312 executes processing of causing the storage unit 32 to store the biological information 321 measured by the measurement unit 41 and received by the communication unit 34. As described later, when surgery is started, the storage processing unit 312 executes processing of causing the storage unit 32 to store the biological information 321 measured by the measurement unit 41 for each process. Furthermore, the storage processing unit 312 executes processing of causing the storage unit 32 to store at least one of the date and time when the surgery is performed, the date and time when the surgery process is performed, or the date and time when the measurement unit 41 measures the biological information 321.

The report generation processing unit 313 executes processing of generating a report based on the biological information 321 stored in the storage unit 32. Examples of the report generated by the report generation processing unit 313 can include, for example, a report in which the surgery processes are arranged from top to bottom or from left to right in time series, and the biological information 321 stored in the storage unit 32 is displayed or described for each process. However, the report generated by the report generation processing unit 313 is not limited to the examples disclosed herein.

For example, the input processing unit 314 executes processing of inputting information input by a medical worker (hereinafter, the medical worker means a medical worker who is given an access right to the biological information storage system 2) who is given an access right to the biological information storage system 2 in response to an operation of the medical worker on an input unit such as the touch panel 33. For example, the input processing unit 314 executes processing of inputting information regarding a patient before the start of the surgery and executes processing of causing the storage unit 32 to store the information. The input processing unit 314 may execute processing of inputting information regarding a patient, and the storage processing unit 312 may execute processing of causing the storage unit 32 to store the information regarding a patient input by the input processing unit 314. Alternatively, for example, the input processing unit 314 executes processing of inputting information regarding a patient before the start of the surgery and executes processing of causing the touch panel 33 to display the information. The input processing unit 314 may execute processing of inputting information regarding a patient, and the display processing unit 311 may execute processing of causing the touch panel 33 to display the information regarding a patient input by the input processing unit 314.

The risk prediction unit 315 can predict a complication risk that will occur after the end of surgery. In the present specification, the “complication” can include at least one of acute kidney injury, acute respiratory distress syndrome, cerebral infarction, non-occlusive mesenteric ischemia, postoperative hypotension, or infection. For example, the risk prediction unit 315 predicts a complication risk that occurs after the end of surgery based on the biological information 321 stored in the storage unit 32 and a trained model 322 stored in advance in the storage unit 32. Alternatively, for example, the risk prediction unit 315 can predict a future event risk that occurs in a case where a scheduled surgery process is performed based on the biological information 321 stored in the storage unit 32. Alternatively, for example, the risk prediction unit 315 can predict a future event risk that occurs in a case where a process different from the scheduled surgery process is performed based on the biological information 321 stored in the storage unit 32.

The notification processing unit 316 executes processing of providing notification of an alert when a predetermined condition is satisfied. For example, in a case where the probability of the complication risk predicted by the risk prediction unit 315 is equal to or greater than a threshold value, the notification processing unit 316 provides notification of an alert on the touch panel 33 and the external monitor 42. The method of notification performed by the notification processing unit 316 may be executed, for example, by generation of light (for example, illuminating a light bulb, illuminating a light-emitting diode (LED), illuminating light, changing dimming of the display unit, or changing color of light) or sound.

The storage unit 32 stores the program 323 executed by a computer including the control unit 31. The program 323 can include a sequence program for measurement, an image processing program for image processing, and an arithmetic program. Examples of the storage unit 32 include a semiconductor memory built in the control system 3. Alternatively, examples of the storage unit 32 include various storage media such as a compact disc (CD), a digital versatile disc (DVD), a random access memory (RAM), a read only memory (ROM), a hard disk, and a memory card, which are connectable to the control system 3, and a data server. The program 323 is not limited to being stored in the storage unit 32, and may be stored in advance in a computer-readable storage medium and distributed, or may be downloaded to the control system 3 via a network.

Furthermore, the storage unit 32 stores the biological information 321 measured by the measurement unit 41 and received by the communication unit 34. For example, the storage unit 32 stores the biological information 321 measured by the measurement unit 41 for each surgery process, that is, in association with the surgery process. The storage unit 32 further stores the trained model 322. The trained model 322 is a model obtained by the control unit 31 performing learning of a prediction algorithm created based on data obtained by a combination of information regarding a patient including the biological information 321 and the past complication that has occurred after the end of surgery.

Next, the operation of the biological information storage system according to the present embodiment, that is, processing executed by the biological information storage system according to the present embodiment will be described with reference to the drawings.

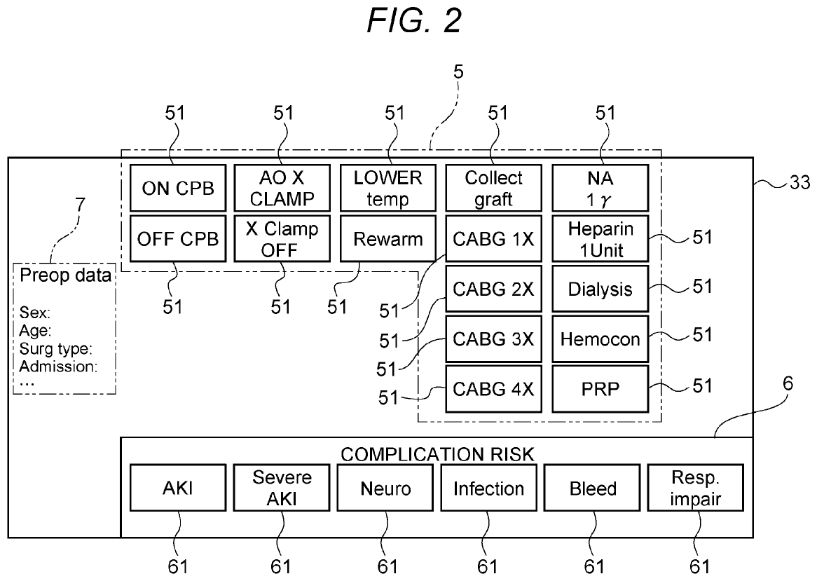

FIGS. 2 to 4 are schematic diagrams illustrating an example of an image displayed on a touch panel of the present embodiment.

Note that as described above, in the description in the present embodiment, the case where the “display unit” of the present disclosure is the touch panel 33 has been described as an example. However, the “display unit” of the present disclosure may be the external monitor 42. That is, examples of the images illustrated in FIGS. 2 to 4 may be displayed on the external monitor 42.

As illustrated in FIG. 2, when the operation of the biological information storage system 2 according to the present embodiment is started, the display processing unit 311 displays a surgery process group 5, a complication risk group 6, and preoperative information 7.

The surgery process group 5 can include a plurality of surgery processes 51 scheduled. In the example of the image illustrated in FIG. 2, a plurality of the surgery processes 51 such as “ON CPB (heart-lung machine operation (cardio pulmonary bypass) ON)”, “OFF CPB (heart-lung machine operation OFF)”, “AO X CLAMP” (aortic cross-clamp ON), “X Clamp OFF” (aortic cross-clamp OFF), “LOWERtemp” (lower temperature), “Rewarm” (rewarming body temperature), “Collect graft” (collect graft), “CABG 1X” (coronary artery bypass grafting single-vessel disease), “Heparin 1 Unit” (injection of one unit of Heparin), and “Dialysis” are displayed on the touch panel 33. The displayed processes are not limited to the processes illustrated, and the arrangement position may be changed by the user, or some of the processes may be configured as a set. For example, in a case where mitral valve replacement is performed, when a set for the mitral valve replacement is selected, general processes are selected at a time. Therefore, a portion to be changed may be selected again in accordance with the day's case and the process set may be optimized in accordance with a doctor in charge or a facility.

The complication risk group 6 can include a plurality of complication risks 61. In the example of the image illustrated in FIG. 2, a plurality of the complication risks 61 such as “AKI” (acute kidney injury), “Severe AKI” (severe acute kidney injury), “Neuro” (neurological impairment), “Infection” (infection), “Bleed” (bleeding), and “Resp. impair” (respiratory impairment) are displayed on the touch panel 33. The notation method is only required to be an easy-to-understand notation. Here, for example, acute kidney injury is described as AKI, bleeding is described as Bleed, and the like, but the notation is not limited to the examples disclosed.

The preoperative information 7 can include information regarding a patient before the start of surgery (Patient background, preoperative examination data, and the like). In the example of the image illustrated in FIG. 2, “Sex”, “Age”, and the like of a patient undergoing surgery are displayed on the touch panel 33. For example, the preoperative information 7 is information input by the medical worker in response to an operation of the medical worker on an input unit such as the touch panel 33, and is information input by the input processing unit 314.

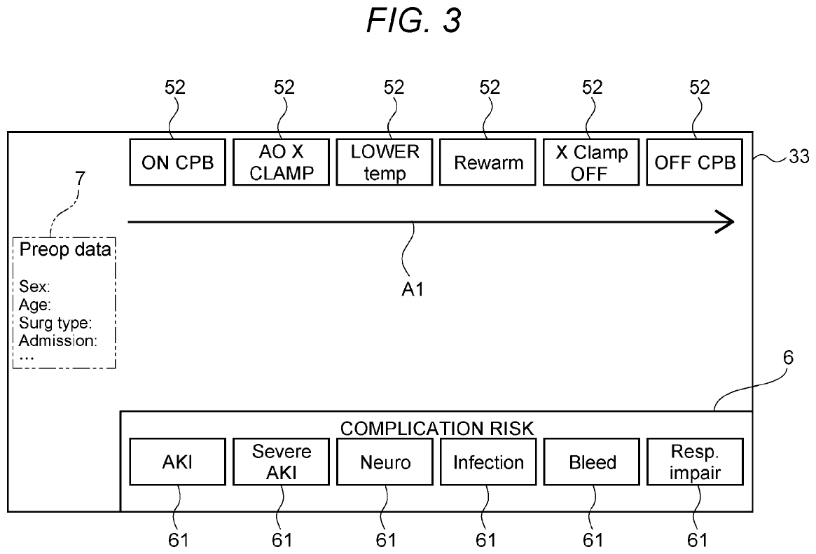

Subsequently, as illustrated in FIG. 3, when the medical workers such as a doctor, a clinical engineer, an anesthesiologist, and a nurse select a plurality of processes from among a plurality of the surgery processes 51 (see FIG. 2) displayed on the touch panel 33 as scheduled surgery processes, the display processing unit 311 arranges a plurality of the selected processes in time series in the surgery procedure and causes the touch panel 33 to display a plurality of the selected processes. In the present embodiment, for convenience of description, a process selected by the medical worker from among a plurality of the surgery processes 51 displayed on the touch panel 33 is referred to as a “selection process”. For example, a selection process 52 (see FIG. 3) is selected by the medical worker in accordance with an operation of the medical worker on an input unit such as the touch panel 33.

In the example of the image illustrated in FIG. 3, as indicated by an arrow A1, a plurality of the selection processes 52 of “ON CPB” (ON cardiopulmonary bypass (heart-lung machine operation)), “AO X CLAMP” (aortic cross-clamp ON), “LOWERtemp” (lower temperature), “Rewarm” (rewarming body temperature), “X Clamp OFF” (aortic cross-clamp OFF), and “OFF CPB” (OFF cardiopulmonary bypass (heart-lung machine operation)) are arranged from left to right in time series in the procedure of surgery and displayed on the touch panel 33 in this order.

A plurality of the selection processes 52 selected by the medical worker may include a predetermined course of surgery. The course of surgery is a framework in which at least some of a plurality of medical acts scheduled to be performed in surgery, techniques, or events are arranged in time series in a procedure of surgery. For example, in a case where it is determined that the scheduled surgery is “CABG-1 branch”, when the medical worker selects “CABG 1X” (see FIG. 2) displayed on the touch panel 33, at least some of a plurality of medical acts set in advance in the surgery course of the “CABG-1 branch”, techniques, and events are automatically arranged in time series in the surgery procedure of the “CABG-1 branch” and displayed on the touch panel 33. Therefore, the medical worker can save time and effort for individually selecting the scheduled surgery processes and rather easily select substantially the entire scheduled surgery processes. Furthermore, the surgery process can be customized according to differences depending on facilities and doctors, and can be registered (for example, the customized surgery process can be registered or saved as a predetermined course of surgery and displayed on the touch panel). The process selected in a case where the scheduled surgery is the “CABG-1 branch” is not limited to the “CABG 1X”.

Furthermore, the display processing unit 311 can add a selection process 52 selected or input later and cause the touch panel 33 to display the selection process 52 in a state in which a plurality of the selection processes 52 are arranged in time series in the procedure of the surgery and displayed on the touch panel 33 as illustrated in FIG. 3. That is, the display processing unit 311 can execute processing of adding the selection process 52 selected or input later to a plurality of the selection processes 52 (see FIG. 3) selected once, rearranging a plurality of the selection processes 52 selected once and the selection process 52 selected or input later in time series in the procedure of surgery, and causing the touch panel 33 to display the rearranged selection processes. Therefore, the biological information storage system 2 according to the present embodiment can flexibly cope with (i.e., have the capacity to incorporate) various surgery processes.

Subsequently, when surgery is started, the storage processing unit 312 causes the storage unit 32 to store the biological information 321 measured by the measurement unit 41 for each selection process 52. Since the technique and procedure in surgery are uniform to some extent, the storage processing unit 312 can automatically cause the storage unit 32 to store the biological information 321 of a patient during the surgery along the flow of the surgery. Furthermore, in a case where there is an additional technique each time, the display processing unit 311 executes processing of adding a freely input process to the procedures arranged in time series. The storage processing unit 312 executes processing of causing the storage unit 32 to store the biological information 321 measured by the measurement unit 41 at that time. In this case, a timing at which the storage processing unit 312 causes the storage unit 32 to store the biological information 321 may be an automatic timing, or may be a timing at which the medical worker designates the selection process 52. Furthermore, the storage processing unit 312 executes processing of causing the storage unit 32 to store at least one of the date and time when the selection process 52 is performed, or the date and time when the measurement unit 41 measures the biological information 321 in the selection process 52.

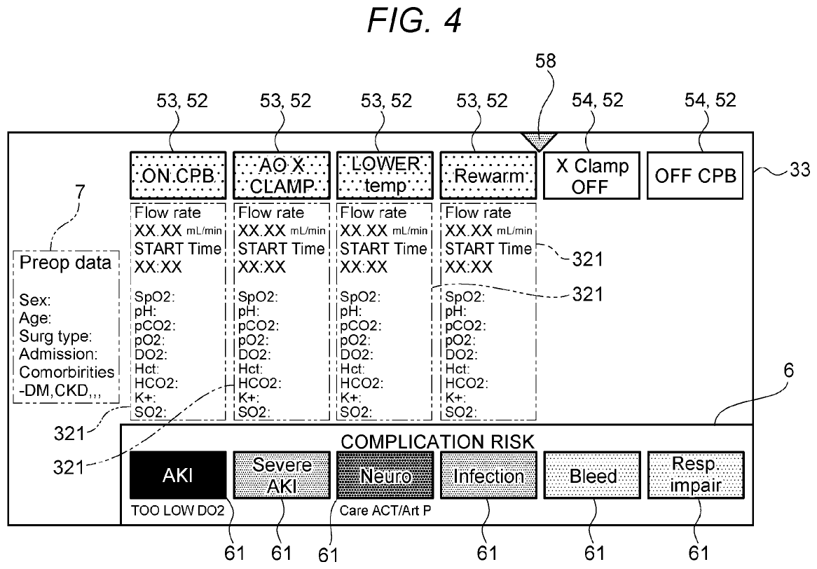

Subsequently, as illustrated in FIG. 4, after the surgery is started, when the medical worker designates an arbitrary process from among the selection processes 52 that have ended in the progress of surgery, the display processing unit 311 reads the biological information 321 in the process designated by the medical worker from the storage unit 32 and causes the touch panel 33 to display the biological information. In the present embodiment, for convenience of description, the process designated by the medical worker from among the selection processes 52 which have ended in the progress of surgery is referred to as a “designation process”. For example, a designation process 53 (see FIG. 4) is designated by the medical worker in accordance with an operation of the medical worker on an input unit such as the touch panel 33.

In the example of the image illustrated in FIG. 4, a plurality of the designation processes 53 of “ON CPB”, “AO X CLAMP”, “LOWERtemp”, and “Rewarm” are designated by the medical worker and highlighted. The biological information 321 corresponding to each of a plurality of the designation processes 53 and stored in the storage unit 32 for each selection process 52 is read from the storage unit 32 by the display processing unit 311 and displayed on the touch panel 33. Therefore, the medical worker can rather easily confirm the biological information 321 in the designation process 53 designated from the ended selection process 52 on the touch panel 33 in a relatively shorter time.

Furthermore, as described above, the preoperative information 7 is displayed on the touch panel 33. Therefore, as illustrated in FIG. 4, the medical worker can rather easily confirm, on the touch panel 33, information obtained by combining information regarding a patient before the start of surgery (that is, the preoperative information 7) and the biological information 321 stored in the storage unit 32 for each selection process 52.

Subsequently, the risk prediction unit 315 can predict a complication risk 61 that occurs after the end of surgery based on the biological information 321 stored in the storage unit 32 for each selection process 52 and the trained model 322 stored in advance in the storage unit 32. In a case where the probability of the risk predicted by the risk prediction unit 315 is equal to or greater than a threshold value, the notification processing unit 316 provides notification, for example, in the form of an alert. In the example of the image illustrated in FIG. 4, “AKI” (acute kidney injury) and “Neuro” (neurological impairment) among a plurality of the complication risks 61 are provided in notification with the alert and highlighted. In this case, the biological information 321 to be noted, for example, may be highlighted.

Therefore, the biological information storage system 2 according to the present embodiment can present the medical worker with the complication risk 61 that occurs after the end of surgery, reduce the burden on the medical worker, and support the medical worker's determination of a complication risk 61 or the medical worker's determinations of medical acts. Furthermore, the medical worker can rather easily recognize that the complication risk 61 that occurs after the end of surgery is relatively high.

Subsequently, the report generation processing unit 313 generates a report based on the biological information 321 stored in the storage unit 32. For example, the report generation processing unit 313 generates a report in which the selection processes 52 can be arranged, for example, from top to bottom or from left to right in time series, and the biological information 321 stored in the storage unit 32 is displayed or described for each selection process 52. The contents and items described in the report may be specifications that can be customized for each facility or by the doctor in charge or the medical worker.

As described above, according to the biological information storage system 2 and the biological information storage program according to the present embodiment, the display processing unit 311 causes the touch panel 33 to display a plurality of the surgery processes 51. When the medical workers, for example, such as a doctor, a clinical engineer, an anesthesiologist, and a nurse select a plurality of the selection processes 52 from among a plurality of the surgery processes 51 displayed on the touch panel 33 as scheduled surgery processes, the display processing unit 311 causes the touch panel 33 to display a plurality of the selected selection processes 52 in time series in the procedure of the surgery. When surgery is started, the storage processing unit 312 causes the storage unit 32 to store the biological information 321 measured by the measurement unit 41 for each selection process 52. Since the technique and procedure in surgery can be uniform to some extent, the storage processing unit 312 can automatically cause the storage unit 32 to store the biological information 321 of a patient during the surgery along the flow of the surgery. Furthermore, in a case where there is an additional technique each time, the display processing unit 311 executes processing of adding a freely input process to the procedures arranged in time series. The storage processing unit 312 executes processing of causing the storage unit 32 to store the biological information 321 measured by the measurement unit 41 at that time. In this case, a timing at which the storage processing unit 312 causes the storage unit 32 to store the biological information 321 may be an automatic timing, or may be a timing at which the medical worker designates the selection process 52. The report generation processing unit 313 can generate a report based on the biological information 321 stored in the storage unit 32. Therefore, the report generation processing unit 313 can help avoid complicated recording work and automatically create a report with a relatively simple operation. Therefore, the biological information storage system 2 according to the present embodiment can help reduce a burden on the medical workers, for example, such as a doctor, a clinical engineer, an anesthesiologist, and a nurse and help prevent them from forgetting to record the biological information 321 of a patient.

Next, a specific example of the operation of the biological information storage system according to the present embodiment, that is, processing executed by the biological information storage system according to the present embodiment will be described with reference to the drawings.

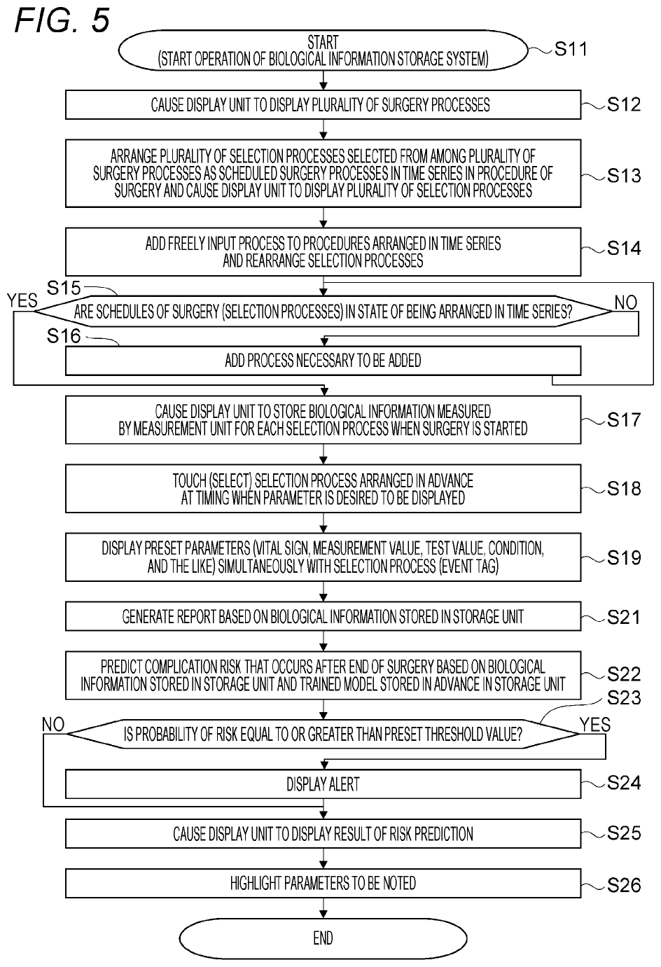

FIG. 5 is a flowchart illustrating a specific example of the operation of the biological information storage system according to the present embodiment.

First, in S11, the medical worker starts the operation of the biological information storage system 2. Then, in S12, the display processing unit 311 causes the display unit (touch panel 33 in the present embodiment) to display a plurality of the surgery processes 51 (see FIG. 2). S12 of the present embodiment is an example of a “first step” of the present disclosure. Subsequently, in S13, the display processing unit 311 arranges a plurality of the selection processes 52 selected from among a plurality of the surgery processes 51 displayed on the touch panel 33 as scheduled surgery processes in time series in the procedure of the surgery and causes the touch panel 33 to display a plurality of the selected surgery processes 51 (see FIG. 3). S13 of the present embodiment is an example of a “second step” of the present disclosure.

Subsequently, in S14, in a case where there is an additional technique each time, the display processing unit 311 executes processing of adding a freely input process to the procedures arranged in time series. That is, in a case where there is the selection process 52 selected or input later for a plurality of the selection processes 52 selected once, the display processing unit 311 adds the selection process 52 selected or input later. Then, the display processing unit 311 executes processing of rearranging a plurality of the selection processes 52 selected once and the selection process 52 selected or input later in time series in the procedure of surgery and causing the touch panel 33 to display the rearranged selection processes.

Subsequently, in S15, the control unit 31 determines whether or not the schedules of surgery (that is, the selection processes 52) are in a state of being arranged in time series. In a case where the schedules of surgery (that is, the selection processes 52) are not in a state of being arranged in time series (S15: NO), in S16, the display processing unit 311 executes processing of adding the necessary selection process 52 added by the medical worker. On the other hand, in a case where the schedules of surgery (that is, the selection processes 52) are in a state of being arranged in time series (S15: YES), in S17, the storage processing unit 312 causes the storage unit 32 to store the biological information 321 measured by the measurement unit 41 for each selection process 52 when the surgery is started. S17 of the present embodiment is an example of a “third step” of the present disclosure.

Subsequently, in S18, the medical worker touches the selection process 52 arranged in advance on the touch panel 33 at a timing when the medical worker desires to display a parameter (that is, the biological information 321). Then, in S19, the display processing unit 311 causes the touch panel 33 to display preset parameters (for example, a vital sign, a measurement value, a test value, a condition, and the like) simultaneously with the selection process 52 touched by the medical worker. That is, in S19, the display processing unit 311 reads the parameters including the biological information 321 in the designation process 53 (see FIG. 4) designated by the medical worker from the storage unit 32 and causes the touch panel 33 to display the parameters.

At this time, the display processing unit 311 causes the touch panel 33 to display biological information 321 of which a value increases as compared with the previous value and biological information 321 of which a value decreases as compared with the previous value, such that the medical worker can rather easily recognize the biological information. For example, the display processing unit 311 causes the touch panel 33 to display the state of change in the biological information 321 by adding a color and an arrow to the biological information 321 or changing the inclination of the arrow according to the rate of change. Furthermore, in a case where there is an item that requires additional display, the display processing unit 311 may additionally cause the touch panel 33 to display the necessary item.

Subsequently, in S20, the report generation processing unit 313 generates a report based on the biological information 321 stored in the storage unit 32. S20 of the present embodiment is an example of a “fourth step” of the present disclosure.

Subsequently, in S21, the risk prediction unit 315 predicts a complication risk 61 that occurs after the end of surgery based on the biological information 321 stored in the storage unit 32 for each selection process 52 and the trained model 322 stored in advance in the storage unit 32. Subsequently, in S22, the notification processing unit 316 determines whether or not the probability of the risk predicted by the risk prediction unit 315 is equal to or greater than a threshold value. In a case where the probability of the risk predicted by the risk prediction unit 315 is equal to or greater than a threshold value (S22: YES), in S23, the notification processing unit 316 causes the touch panel 33 to display an alert. On the other hand, in a case where the probability of the risk predicted by the risk prediction unit 315 is not equal to or greater than a threshold value (S22: NO), the notification processing unit 316 does not cause the touch panel 33 to display the alert.

Subsequently, in S24, the display processing unit 311 causes the touch panel 33 to display a result of the risk predicted by the risk prediction unit 315. Furthermore, in S25, the display processing unit 311 highlights parameters to be noted.

Next, a specific example of processing executed by the risk prediction unit 315 of the present embodiment will be described with reference to the drawing.

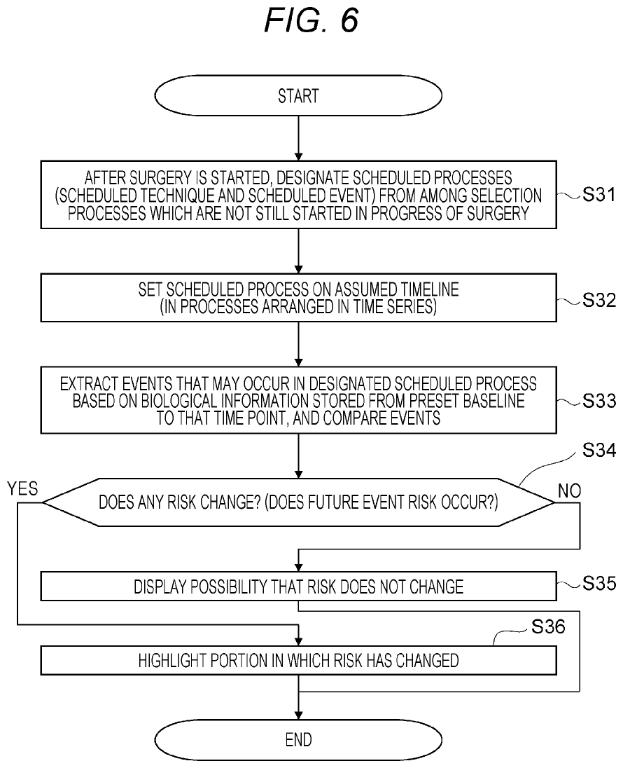

FIG. 6 is a flowchart illustrating a first specific example of processing executed by the risk prediction unit of the present embodiment.

In the present specific example, after the surgery is started, the risk prediction unit 315 predicts a future event risk that occurs in a case where the process designated from among the selection processes 52 (see FIG. 4) which are not still started in the progress of surgery is performed based on the biological information 321 stored in the storage unit 32. In the present embodiment, for convenience of description, the process designated by the medical worker from among the selection processes 52 which are not still started in the progress of surgery is referred to as a “scheduled process”. For example, a scheduled process 54 (see FIG. 4) is designated by the medical worker in accordance with an operation of the medical worker on an input unit such as the touch panel 33.

In the description using the example of the image illustrated in FIG. 4, the current time point in a plurality of the selection processes 52 during the surgery is indicated by a triangle mark 58. In the example of the image illustrated in FIG. 4, the selection processes 52 which are not still started in the progress of surgery are “X Clamp OFF” and “OFF CPB”. Then, when the medical worker designates at least one of “X Clamp OFF” or “OFF CPB” as the scheduled process 54, the risk prediction unit 315 predicts a future event risk that occurs in a case where the scheduled process 54 is performed based on the biological information 321 stored in the storage unit 32.

That is, in S31, after the surgery is started, the medical worker designates a scheduled process 54 from among the selection processes 52 which are not still started in the progress of surgery. The “scheduled process” is also referred to as a “scheduled technique” or a “scheduled event”. Subsequently, in S32, the display processing unit 311 sets the scheduled process 54 on an assumed timeline (that is, in the processes arranged in time series) and causes the touch panel 33 to displays the scheduled process.

Subsequently, in S33, the risk prediction unit 315 extracts events that may occur in the designated scheduled process 54 based on the biological information 321 stored in the storage unit 32 from a preset baseline (that is, data at a reference time point) to the time point, and compares the events. Subsequently, in S34, the risk prediction unit 315 determines whether or not a certain risk changes, that is, whether or not a future event risk occurs.

In a case where any risk does not change (S34: NO), in S35, the display processing unit 311 causes the touch panel 33 to display the possibility that the risk does not change. In a case where any risk changes (S34: YES), in S36, the display processing unit 311 highlights a portion in which the risk has changed.

According to the specific example, the biological information storage system 2 can present a future event risk that occurs in a case where the scheduled process 54 scheduled to be performed along the flow of the selection process 52 is performed, reduce a relative burden of recording the biological information of the patient on the medical worker, and support the medical worker's determinations of medical acts.

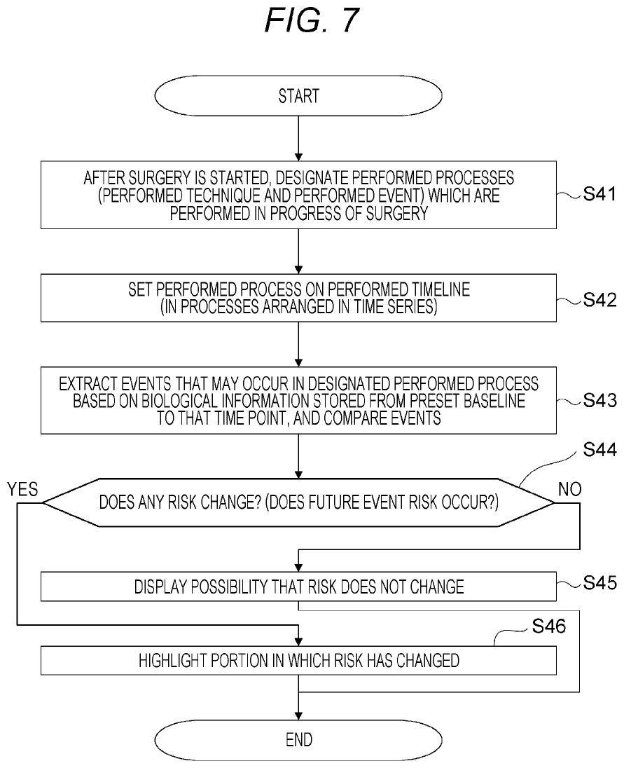

FIG. 7 is a flowchart illustrating a second specific example of processing executed by the risk prediction unit of the present embodiment.

In the present specific example, after the surgery is started, the risk prediction unit 315 predicts a future event risk that occurs in a case where the process designated from among the selection processes 52 which are performed in the progress of surgery is performed based on the biological information 321 stored in the storage unit 32. In the present embodiment, for convenience of description, the process designated by the medical worker from among the selection processes 52 which has performed in the progress of surgery is referred to as a “performed process”. The “performed process” is also referred to as a “performed technique” or a “performed event”.

In the description using the example of the image illustrated in FIG. 4, the selection processes 52 performed in the progress of surgery can be displayed on the left side of the triangle mark 58 illustrated in FIG. 4. Furthermore, the performed processes designated from among the selection processes 52 performed in the progress of surgery correspond to the designation processes 53 described in FIG. 4. As described in FIG. 4, for example, the designation process 53 is designated by the medical worker in accordance with an operation of the medical worker on an input unit such as the touch panel 33.

First, in S41, after the surgery is started, the medical worker designates a performed process from among the selection processes 52 which are performed in the progress of surgery. Subsequently, in S42, the display processing unit 311 sets a performed process on an assumed timeline (that is, in the processes arranged in time series) and causes the touch panel 33 to displays the performed process.

Subsequently, in S43, the risk prediction unit 315 extracts events that may occur in the designated performed process based on the biological information 321 stored in the storage unit 32 from a preset baseline (that is, data at a reference time point) to the time point, and compares the events. Subsequently, in S44, the risk prediction unit 315 determines whether or not any risk changes, that is, whether or not a future event risk occurs.

In a case where any risk does not change (S44: NO), in S45, the display processing unit 311 causes the touch panel 33 to display the possibility that the risk does not change. On the other hand, in a case where any risk changes (S44: YES), in S46, the display processing unit 311 highlights a portion in which the risk has changed.

According to the specific example, the biological information storage system 2 can present a future event risk that occurs after the performed process performed in the progress of surgery is performed, reduce a relative burden on the medical worker, and support the medical worker's determination.

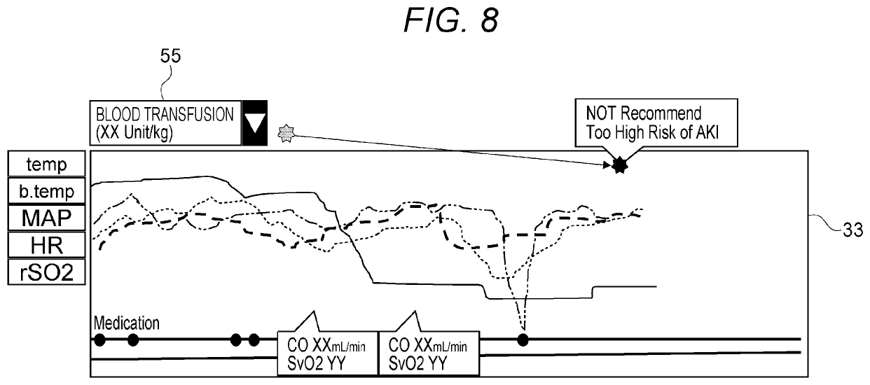

FIG. 8 is a schematic diagram illustrating a third specific example of processing executed by the risk prediction unit of the present embodiment.

Note that in the specific example, FIG. 8 is schematic diagram illustrating an example of an image displayed on the touch panel of the present embodiment.

In the specific example, after the surgery is started, the risk prediction unit 315 predicts a future event risk that occurs in a case where an unscheduled process different from the selection process 52 is performed based on the biological information 321 stored in the storage unit 32. In the present embodiment, for convenience of description, the unscheduled process different from the selection process 52 selected as a scheduled surgery process by the medical worker is referred to as an “unscheduled process”. That is, the unscheduled process is an additionally necessary medical action/process. In the example of the image illustrated in FIG. 8, “blood transfusion” is displayed as an unscheduled process 55.

As described in FIG. 8, after the surgery is started, the risk prediction unit 315 predicts a future event risk that occurs in a case where the blood transfusion as the unscheduled process 55 is performed based on the biological information 321 stored in the storage unit 32. Then, the display processing unit 311 highlights a parameter to be noted based on the future event risk predicted by the risk prediction unit 315.

According to the specific example, the biological information storage system 2 can present a future event risk that occurs in a case where the unscheduled process 55, which is not scheduled to be performed along the flow of the selection process 52, is performed, reduce a relative burden on the medical worker, and support the medical worker's determination.

The embodiments of the present disclosure have been described above. However, the present disclosure is not limited to the above-described embodiments, and various modifications can be made without departing from the scope of the claims. The configurations of the above-described embodiments can be partially omitted, or can be arbitrarily combined so as to be different from the above-described configurations.

The detailed description above describes embodiments of a biological information storage system and a biological information storage program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A biological information storage system comprising:
a measurement unit configured to measure biological information of a patient undergoing surgery;
a storage unit configured to store the biological information measured by the measurement unit;
a display unit configured to display the biological information;
a display processing unit configured to execute processing of causing the display unit to display a plurality of surgery processes, to arrange a plurality of selection processes selected from among the plurality of surgery processes as the scheduled surgery processes in time series in a procedure of the surgery, and to cause the display unit to display the plurality of selection processes;
a storage processing unit configured to execute processing of causing the storage unit to store the biological information measured by the measurement unit for each of the selection processes when the surgery is started; and
a report generation processing unit configured to execute processing of generating a report based on the biological information stored in the storage unit.

2. The biological information storage system according to claim 1, wherein the display processing unit is configured to:
execute processing of adding the selection process selected or input later to the plurality of selection processes selected;
rearrange the plurality of selection processes selected once and the selection process selected or input later in time series in the procedure of the surgery; and
cause the display unit to display the rearranged selection processes.

3. The biological information storage system according to claim 1, wherein after the surgery is started, the display processing unit is configured to execute processing of causing the display unit to display the biological information in a designation process designated from among the selection processes that have ended in a progress of the surgery.

4. The biological information storage system according to claim 1, wherein the plurality of selection processes include a predetermined course of the surgery.

5. The biological information storage system according to claim 1, further comprising:
an input processing unit configured to execute processing of inputting information regarding the patient before the surgery is started.

6. The biological information storage system according to claim 1, further comprising:
a risk prediction unit configured to predict a risk of a complication that occurs after the surgery is ended based on the biological information stored in the storage unit and a trained model stored in advance in the storage unit.

7. The biological information storage system according to claim 6, wherein the complication includes at least one of acute kidney injury, acute respiratory distress syndrome, cerebral infarction, non-occlusive mesenteric ischemia, postoperative hypotension, or infection.

8. The biological information storage system according to claim 6, wherein after the surgery is started, the risk prediction unit is further configured to predict, based on the biological information stored in the storage unit, a future event risk that occurs in a case where a scheduled process designated from among the selection processes which are not still started in a progress of the surgery is performed.

9. The biological information storage system according to claim 6, wherein after the surgery is started, the risk prediction unit is further configured to predict, based on the biological information stored in the storage unit, a future event risk that occurs in a case where an unscheduled process different from the selection process is performed.

10. The biological information storage system according to claim 6, further comprising:
a notification processing unit configured to execute processing of providing notification of an alert in a case where a probability of the risk predicted by the risk prediction unit is equal to or greater than a threshold value.

11. A non-transitory computer-readable medium storing a biological information computer program executed by a computer of a biological information storage system including a measurement unit configured to measure biological information of a patient undergoing surgery, a storage unit configured to store the biological information measured by the measurement unit, and a display unit configured to display the biological information, the biological information computer program causing the computer to execute a process comprising:
causing the display unit to display a plurality of surgery processes;
arranging a plurality of selection processes selected from among the plurality of surgery processes as the scheduled surgery processes in time series in a procedure of the surgery;
causing the display unit to display the plurality of selection processes;
causing the storage unit to store the biological information measured by the measurement unit for each of the selection processes when the surgery is started; and
generating a report based on the biological information stored in the storage unit.

12. The computer-readable medium according to claim 11, further comprising:
adding the selection process selected or input later to the plurality of selection processes selected;
rearranging the plurality of selection processes selected once and the selection process selected or input later in time series in the procedure of the surgery; and
causing the display unit to display the rearranged selection processes.

13. The computer-readable medium according to claim 11, wherein after the surgery is started, the display processing unit is configured to execute processing of causing the display unit to display the biological information in a designation process designated from among the selection processes that have ended in a progress of the surgery.

14. The computer-readable medium according to claim 11, wherein the plurality of selection processes include a predetermined course of the surgery.

15. The computer-readable medium according to claim 11, further comprising:
execute processing of inputting information regarding the patient before the surgery is started from an input processing unit.

16. The computer-readable medium according to claim 11, further comprising:
predicting a risk of a complication that occurs after the surgery is ended based on the biological information stored in the storage unit and a trained model stored in advance in the storage unit.

17. A method for storing biological information comprising:
measuring biological information of a patient undergoing surgery;
storing the measured biological information;
displaying the biological information and a plurality of surgery processes on a display unit;
arranging a plurality of selection processes selected from among the plurality of surgery processes as the scheduled surgery processes in time series in a procedure of the surgery;
displaying the plurality of selection processes on the display unit;
storing the measured biological information of plurality of the selection processes when the surgery is started; and
generating a report based on the stored biological information.

18. The method according to claim 17, further comprising:
adding the selection process selected or input later to the plurality of selection processes selected once;
rearranging the plurality of selection processes selected and the selection process selected or input later in time series in the procedure of the surgery; and
displaying the rearranged selection processes on the display unit.

19. The method according to claim 17, wherein after the surgery is started, the method further comprises:
displaying the biological information on the display unit in a designation process designated from among the selection processes that have ended in a progress of the surgery.

20. The method according to claim 17, further comprising:
predicting a risk of a complication that occurs after the surgery is ended based on the biological information stored in the storage unit and a trained model stored in advance in the storage unit.
